(19) United States
(12) Patent Application Publication
(10) Pub. No.: US 2006/0049046 A1
(43) Pub. Date: Mar. 9, 2006

(54) Flexible biochip

(75) Inventors: Marc Cuzin, Corenc (FR); Michel Guy, Grenoble (FR); Philippe Cleuziat, Isle d'abeau (FR)

Correspondence Address: OLIFF & BERRIDGE, PLC, P.O. BOX 19928, ALEXANDRIA, VA, 22320, US

(73) Assignee: APIBIO, Marcy L'Etoile (FR)

(21) Appl. No.: 10/520,105

(22) PCT Filed: Jul. 18, 2003
(86) PCT No.: PCT/FR03/02287

§ 371 Date: Jan. 11, 2005

(30) Foreign Application Priority Data
Jul. 19, 2002 (FR) 0209232

(51) Int. Cl. G01N 33/487; G01N 27/26

(52) U.S. Cl. 204/403.01; 205/775

(57) ABSTRACT

The invention concerns a sensor (1), in particular a biosensor, comprising an electrically or electronically insulating support (2), including at least one active surface (20), a plurality of electrically or electronically conductive electrodes (31,32), arranged on the active surface (2a) of the support in a predetermined operative arrangement, exposed, that is said electrodes can be jointly placed in contact with a common external medium, for example a liquid medium; a plurality of electric terminals (4) respectively corresponding to said electrodes (4), arranged on the active surface (2a, 2b) of the support, exposed, that is said terminals can be electrically or electronically connected outside, independently of one another; a plurality of electrically or electronically conductive strips (5), extending along one (2a) and/or the other (2d) of the support surfaces, connecting the plurality of electrodes (31,32) respectively to the plurality of terminals (4); an electrically or electronically insulating material layer (6), coating one (2a) and/or the other (2b) of the support surfaces, covering at least part of said strip conductors and exposing both the electrodes (31,32) and the terminals (5). The invention is characterized in that, in combination, the plurality of electrodes (4) is arranged in an end zone (1a) opposite another end zone wherein electric terminals (5) are assembled together and the support (2) comprises at least one flexible zone (1c), located between the two end zones.

The present invention relates to a sensor, and more particularly to a biosensor, for use as a tool in molecular biological analysis.

The term “biosensor” is understood to mean a functional assembly, for one-time use or not, for example use for the purpose of a molecular biological determination, designed and/or intended to cooperate with at least one separate and complementary apparatus or instrument that receives a liquid or fluid, immobile or moving, specimen of interest, said liquid specimen comprising at least one target species, in suspension or in solution, for example an optionally labeled biomolecule; and the biosensor delivers at least one output signal relating to the presence and/or the nature and/or the structure and/or the quantity of said target species. As regards a small biosensor, for example of the order of one centimeter, this may be called a “biochip” according to the terminology adopted in the technical field in question.

The biosensor comprises at least:
a support having a useful face comprising an operating surface in contact with the specimen; the material or matter of the support is inert and is substantially electrically nonconductive, in the sense that there is practically no interaction between said material and the specimen, and in particular the target species, by a strong bond of the covalent chemical bond type, or by a weak bond, for example a hydrogen bond, other interactions of a physical type, such as surface tension, not however being excluded; the constituent material of such a support is, for example a plastic, for example a thermoplastic resin, for example a polypropylene;
an operating arrangement of elementary sites distributed in a predetermined manner over the operating surface, each elementary site being addressed, that is to say identified by coordinates that are unique to it; these elementary sites are themselves optionally treated in order to form electrodes, for the purpose of attaching or anchoring the ligands, which will be discussed later; this treatment may for example consist of a coating with a layer of an electronically conductive polymer, for example a modified polypyrrole, according to the techniques disclosed in documents FR 2 703 359, WO 94/22889, EP 0 691 978, FR 2 787 582, EP 1 141 391 and WO 00/36145; when this treatment is completed, what is obtained on the support is a multiplicity of electrically or electronically conducting electrodes that are placed on the useful face of the support in the operating arrangement adopted, and are exposed in the sense that these electrodes may be brought together into contact with one and the same external liquid medium, in this case the specimen of interest;
a set of connections, or circuit, of electrical or electronic type, with the various elementary electrodes respectively, said set of connections being designed for individually connecting each elementary electrode independently of the other elementary electrodes, for example by addressing (see document FR-A 2 741 475); a multiplicity of electrical terminals, corresponding to the aforementioned electrodes respectively, which are placed on a useful face of the support and are exposed in the sense that said terminals may be electrically or electronically connected to the outside independently of one another; a multiplicity of electrically or electronically conducting tracks, each running on one of the faces of the support and/or the other, connecting the multiplicity of electrodes to the multiplicity of terminals respectively; said set of connections is therefore a multiplexed electrical or electronic circuit of electrical terminals and of electrodes and counterelectrodes that are connected respectively to them; and
a layer of an electrically or electronically insulating material, for example a lacquer, coating one face of the support and/or the other, on the one hand at least partly covering said tracks and on the other hand exposing both the electrodes and the terminals.

The dimensions of these biosensors, for example, from around 1 mm² to a few cm², may require the use of “micro” techniques or “nanotechnologies”, for example lithography or micromachining, in order to produce them.

However, the Applicant does not intend to be limited to particular dimensions, for example of the order of 1 μm or 1 nm, when the term “sensor”, “biosensor” or “biochip” is used in the present description and in the appended claims, considering that the same structure or the same arrangement as that defined below may be used with dimensions of the order of a few mm², just as with much larger dimensions.

Of course, a biosensor as considered by the present invention does not operate autonomously, unless its own power supply is incorporated with it. Consequently, this biosensor is designed to cooperate, for example, in a removable manner, on the one hand with external means for making the liquid specimen of interest, but also other fluids or liquids such as a washing liquid, circulate or remain in contact with the operating surface and with the ligands, and on the other hand with means for detecting and for processing the output signal or signals, all this being in general monitored and controlled by external, analog or computer, electronic means, for example, according to any processing flowchart or software.

The term “biomolecule” is understood to mean any entity, in particular a biochemical or biological entity, identical to or derived from any molecular species existing in nature. Among the biomolecules considered by the present invention, mention may be made of certain biopolymers, for example DNA and RNA, oligonucleotides and polynucleotides, functional or structural proteins, peptides, oligopeptides and polypeptides, polysaccharides, etc.

The term “labeling” or “labeled” is understood to mean the characteristic whereby a label is attached to an entity, for example the target species, in a covalent or other manner, said label being a substituent or residue for producing a signal, referred to above as the output signal, with or without the aid of an external means, such as illumination, and with or without a subsequent step, such as one of contacting it with a substrate.

The preferred labels according to the present invention are:
haptenes, for example biotin attaching the streptavidin-phycoerythrin conjugate;
fluorophores, for example fluorescein, cyanine and phycoerythrin;
luminophores: luminol, isoluminol, ABEI (N-4-aminobutyl-N-ethylisoluminol); and
enzymes, for example for the oxidation of a chromogen; see horseradish peroxydase, alkaline phosphatase.

The term “determination” is understood to mean the qualitative and/or quantitative identification, the detection, the description (for example sequencing), the separation or the enrichment of the target species, which may be called the “analyte” in the case of a qualitative and/or quantitative identification. According to the present invention, the term “determination” includes any sequencing of a biomolecule of the DNA or polypeptide type.

The output signal or signals for the purposes of determination may be of any appropriate type, depending on the labels used, and on the type of detection required. They may be visible or invisible light signals, electrical signals, electrooptic signals, electrochemical signals, etc. Moreover, these signals may where appropriate be detected separately, taking into account, on the one hand, the addressing of the biosensor electrodes and, on the other hand, the set of connections of the electrical terminals to the various respective electrodes present on the biosensor.

The term “ligand” is understood to mean any cellular or biological entity, or biomolecule, having a specific or nonspecific affinity for a target species. Affinity means that it forms, under the conditions (especially temperature, pH, ionic force, etc.) in which the target species is brought into contact with the ligand, a stable complex or pairing between said target species and said ligand. As an example of a ligand, mention may be made of any oligonucleotide capable of binding via weak bonds—in this case we speak of hybridization with a DNA strand (target species) having a sequence complementary to that of the ligand.

Each ligand is attached or anchored at each site or on each electrode of the biosensor, possibly after functionalization of the elementary sites of the operating surface of the support by any suitable means, for example chemical means, by covalent bonding, for example via a spacer arm, or by adsorption, absorption, etc.

With regard to the elementary sites coated, as indicated above, with a polymer of the polythiophene or modified polypyrrole type and electrically addressed, the ligands may be fixed using the electrochemical techniques described in documents FR 2 789 401, EP 1 152 821, WO 00/47317, FR 2 742 451, EP 0 868 464 and WO 97/22648.

The term “target species” is understood to mean any biological or biochemical cell species capable of being bonded via a weak bond to one or more ligands.

With regard to a biosensor of the biochip type, in the current state of the art in the technical sector in question, a distinction may be made between two routes of obtaining respectively different ligands, each multiply attached to the various electrodes respectively:
an in situ route, which consists, by a series of successive incremented operations, in synthesizing, on the operating surface itself, the various ligands, together, elementary unit by elementary unit, for example bit by bit, from a first unit attached at various elementary sites, and in the order of the respective sequences adopted for the various ligands; in this regard the reader may refer to documents FR 2 703 359, EP 0 691 978, WO 94/22889, U.S. Pat. No. 5,744,305, U.S. Pat. No. 6,015,880, WO 95/25116 and EP 0 750 629; and
an ex situ route, which consists in synthesizing or obtaining the respective different ligands away from the operating surface and in attaching the various ligands, each multiply, in their respectively different elementary sites by differentiated (since addressed) electrical activation of said elementary sites; see documents FR 2 703 359, EP 0 691 978, WO 94/22889, WO 01/51689 and U.S. Pat. No. 6,090,933.

At the present time, biosensors of the biochip type are simple or complex tools that are well suited to all kinds of analysis in molecular biology, see “DNA chips: a new tool for genetic analysis and diagnostics” by M. Cuzin, Transfusion Clinique et Biologique 2001; 8:291-6; and “How to make a DNA chip” by Michael C. Pirrung, Angew. Chem. Int. Ed 2002.41, 1276, 1289.

In general, a biosensor as described above is placed at the bottom of a well, a microtitration plate, within which the liquid specimen comprising the target species is introduced, resides and from which it is then removed.

The subject of the present invention is a sensor, in particular a biosensor, for example a biochip, which is particularly simple to manufacture or produce and can be used in the most varied of ways, for example in the wells of a microplate.

According to the present invention on the one hand the multiplicity of electrodes is placed in an extreme zone on the opposite side from another extreme zone in which the electrical terminals are grouped together, and on the other hand the support includes at least one flexible zone located between the two extreme zones.

Preferably, the entire support is flexible and produced, for example, from a thin, flexible insulating material.

The term “flexible” is understood to mean in particular that the zone of the same name can bend about at least one axis having a direction perpendicular to the direction of alignment of the operating arrangement of the electrodes and of the group of electrical terminals.

Preferably, the support is a flexible sheet or plate made of insulating material.

Thanks to the present invention, the purely electrical zone of the sensor is shifted relative to its active zone, that is to say that zone having the electrodes and grouping them together, to which the ligands are respectively attached, and this is done by allowing any relative position between the two extreme zones having the electrical terminals and the electrodes respectively, thanks to the flexibility of the support, at least in its intermediate zone between the two said extreme zones.

Thus, the electrodes may be immersed in a liquid solution, whereas the electrical terminals are in the open air and/or in the dry. This makes it possible to achieve, under excellent technical or practical conditions:
the addressing of the ligands in an extreme zone, with a measurement by electrical detection in another extreme zone;
quality control during the step of addressing the probes or ligands on their respective electrodes and the hybridization step (pairing of the ligands attached to the support with the one or more target species of interest), for example by measuring the impedance at each binding site—the impedance measurement is different at each site depending on whether or not it is addressed and whether or not it is hybridized; and
with regard to a titration microplate, the separation between the extreme zones makes it possible to place a sensor according to the invention in each well, with its active zone, that is to say the zone having the electrodes, at the bottom of said well and its electrical zone, that is to say the zone having the electrical terminals, on the periphery, for example on the edge of the microplate.

A sensor according to the invention makes it possible to fabricate and employ biochips in a completely different approach from the conventional approach, by:
fabricating the biochips on one and the same flexible support, and then cutting this up;
transferring each cut biochip to the place of its use, for example at the bottom of a well of a microtitration plate, by being adapted to the conformation of the place of use.

The present invention will now be described with reference to the appended drawing, in which:

FIG. 1 shows, on an enlarged scale, a biosensor of the biochip type according to the present invention;

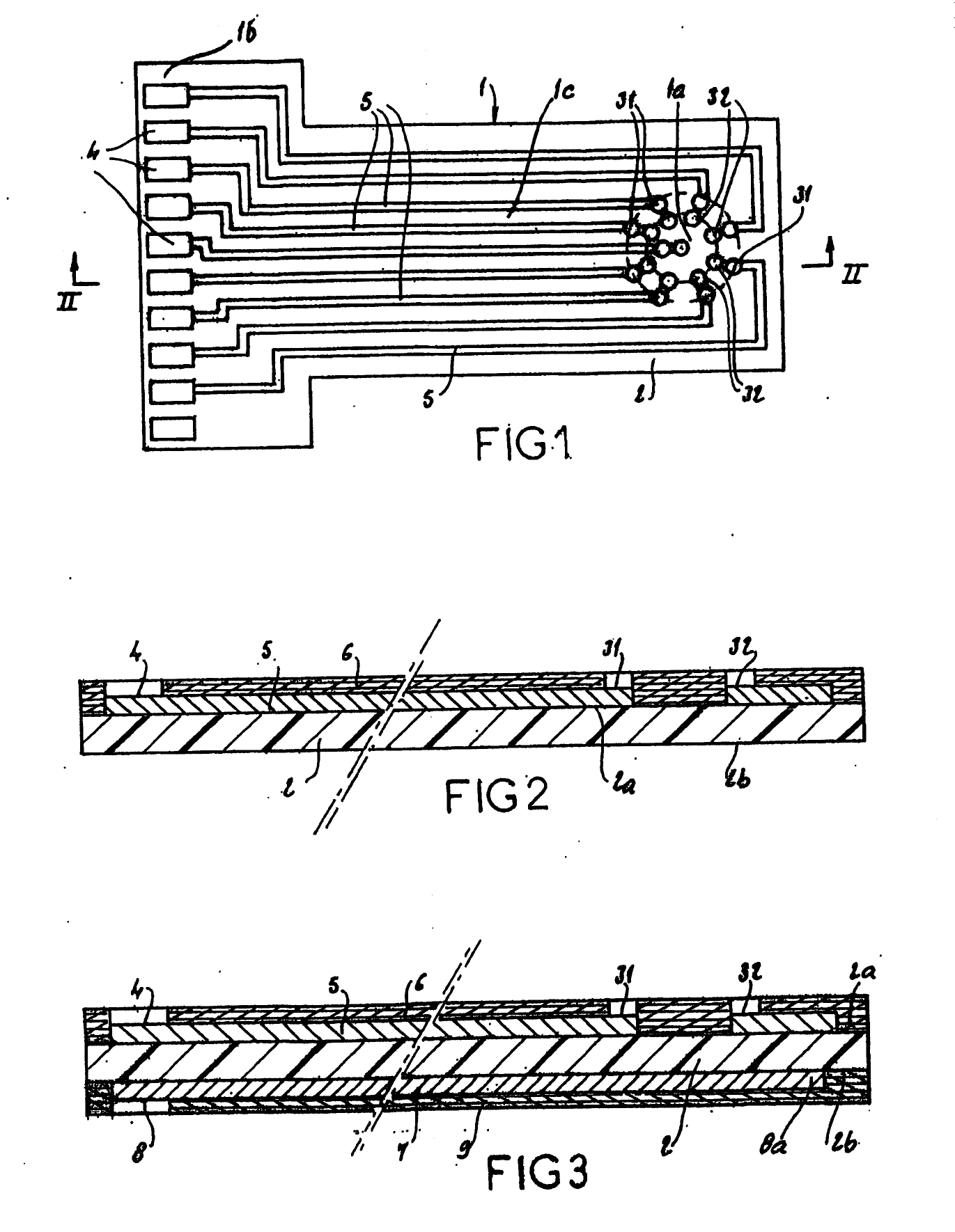

FIG. 2 shows a sectional view on the line II/II of FIG. 1, schematically, of the biochip shown in FIG. 1;

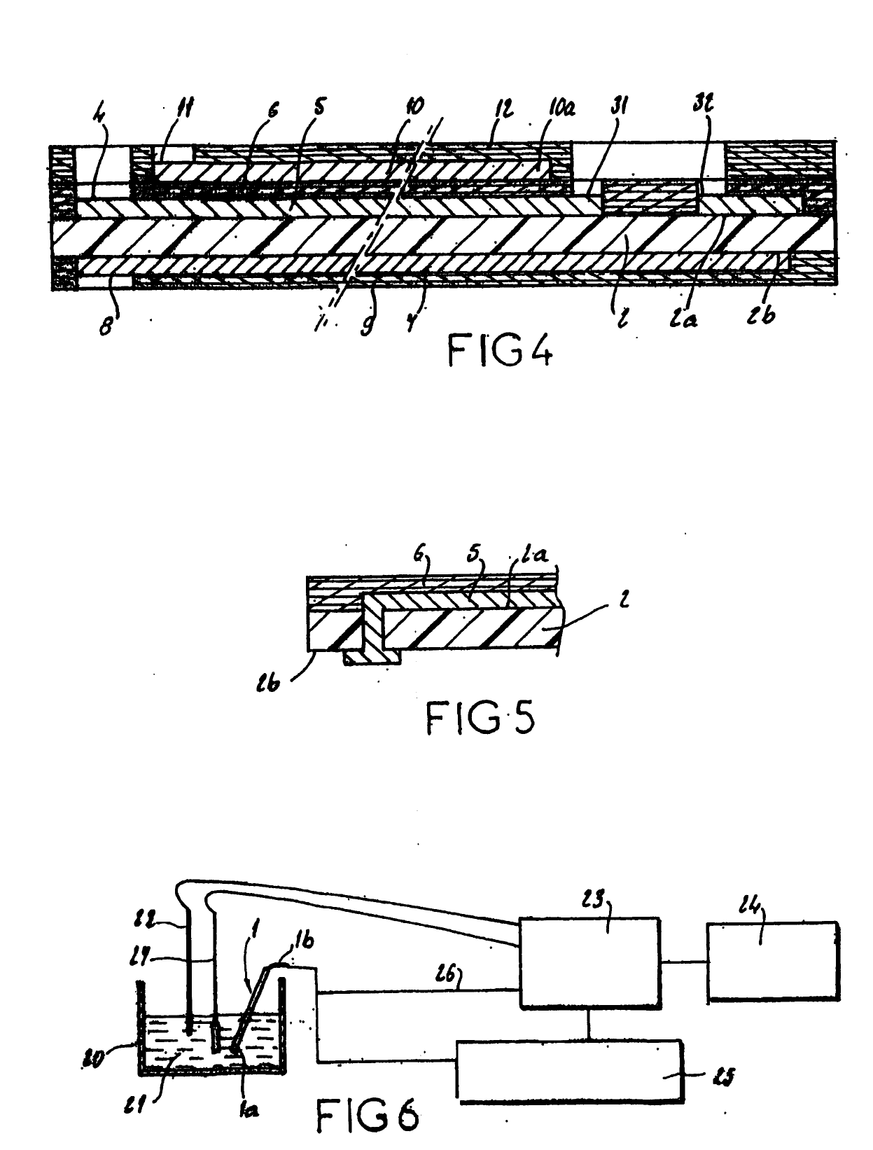

FIGS. 3 and 4 show two other embodiments of the present invention, respectively; and

FIG. 5 shows a detail (at the left-hand end in the representation shown in FIG. 2) of one way of executing another embodiment of the present invention.

According to FIGS. 1 and 2, a sensor (1) in particular, a biosensor according to the present invention, comprises:
an electrically or electronically insulating support (2), for example made of polyimide, comprising a useful face (2a), corresponding to the upper face, and another face (2b) that can be used, corresponding to the lower face;
a multiplicity of electrodes (31, 32), each electrode (31) being connected to an electrode (32), or having at least two adjacent ends (31), (32) connected together, which are electrically or electronically conducting and are placed on the useful face (2a) of the support in any predetermined operating arrangement, and are exposed in the sense that said electrodes may be brought together into contact with one and the same external medium, for example a liquid specimen of interest;
a multiplicity of electrical terminals (4), corresponding to said electrodes (31) respectively, which are placed on a useful face (2a or 2b) of the support (2) and are exposed in the sense that these terminals may be electrically or electronically connected to the outside independently of one another;
a multiplicity of electrically or electronically conducting tracks (5) produced for example in a nickel/gold alloy or a copper/gold alloy, each running along one (2a) of the faces of the support (2) and/or the other (2b), connecting the multiplicity of electrodes (31 and 32) to the multiplicity of terminals (4) respectively; and
a layer (6) of an electrically or electronically insulating material, for example a lacquer, coating one (2a) face of the support (2) and/or the other (2b), on the one hand at least partly, or otherwise completely covering the tracks (5) and on the other hand exposing both the electrodes (31, 32) and the terminals (5).

According to the invention, in combination, on the one hand the multiplicity of electrodes (4) is placed in a zone (1a) on the opposite side from a zone (1b) in which the multiplicity of electrical terminals (5) are grouped together and, on the other hand, the support (2) owing to the flexibility of the insulating material employed, is designed to be flexible at least in an intermediate zone (1c), at least about at least one axis having a direction perpendicular to the direction of alignment of the operating arrangement of the electrodes (31 and 32) in the zone (1a) and of the group of electrical terminals (5) in the zone (1b).

Each assembly, composed of an electrode (31) and an electrode (32), may be seen as one and the same electrode having two adjacent ends connected together.

According to the embodiment shown in FIG. 3, another electrically or electronically conducting track (7) runs along the other face (2b) of the support (2), from another electrical terminal (8) placed on the face (2b) of the support thus made useful; this terminal (8) is exposed in order to be connected to a reference potential and one end (8a) of the track (7) is covered with a layer (9) of the electronic insulating material, for example a lacquer.

Of course, the arrangement described above could be applied on the useful face (2a).

According to the embodiment shown in FIG. 4, two other electrically or electronically conducting tracks (7) and (10) run between two other respective electrical terminals (8 and 11) in order to be connected to a reference potential, these being placed on one face (2a) of the support (2) and on the other face (2b) respectively, and two respective ends (8a) and (10a) that are each covered with the electrically or electronically insulating material, for example a lacquer.

The track (7) and/or the track (10) thus described may be assigned to the electrical shielding of the arrangement of the electrodes (31 and 32) so as to prevent any electromagnetic radiation from interfering with the address or measurement signals.

As shown in FIG. 5, at lest one electrical terminal (4) is placed on the other face (2b) of the support (2), this also being a useful face within the context of the present invention and the track (5) that corresponds to it passes through the thickness of the support (2). This arrangement makes it possible to make electrical contact on the opposite side from the pad (31), in such a way that the latter, optionally in contact with a liquid, is electrically accessible in a zone outside said liquid.

With regard to a biosensor, of the biochip type, a plurality of ligands are each multiply attached to the respectively different electrodes (31 and/or (32).

### EXAMPLE

A sensor, or “flexichip” according to the invention is employed, before or after addressing, or before or after hybridizing, as shown schematically in FIG. 6, with the following numerically referenced members:
1) sensor;
20, 21) a container, in which a liquid (21) is placed;
22) reference electrode;
23) potentiostat;
24) monitoring and control member;
25) supply module;
26) ground;
27) counterelectrode.

A flexichip according to the invention is addressed as follows:
a flexichip (1) is immersed in a copolymerization solution (21) containing 20 mM of pyrrole and 1 μM of ODN1-pyrrole (1 unit of ODN-pyrrole per 20 000 pyrrole units). A potential of +1 V(SCE) is applied for a time t needed for passing a quantity of charge equal to 2.7 mC.cm⁻² per contact pad;
the flexichip (1) is removed and rinsed with deionized water if it is desired to address the other contact pads via another ODN (ODN=oligonucleotide);
next, the flexichip (1) is reimmersed in a solution (21) containing the same pyrrole/ODN-pyrrole molar ratio, but with a different ODN (ODN2); and
these operations are repeated n times, n being the number of contact pads (31, 32), until they have all been addressed.

The nucleotide sequences of the probes deposited are, for example:

| | | |
|---|---|---|
| ODN 1 | ACT-908P | TTTTTTTTTTCTCCACCACTGCTGAAAGAGAAATTGTCCGTGTCATCAAGGAAAAACTAT |
| ODN 2 | GRE1-88P | TTTTTTTTTTAGACAACAGCGTCATGAAAAACATCAACAGAGGGAATTCAGGAATCAAGG |
| ODN 3 | KRR1-456P | TTTTTTTTTTTTTAAAGGCTTTGGAACTTCTAACTAAATGTTACATTCTAGTACAAGGTA |
| ODN 4 | RPS31-246P | TTTTTTTTTTGAAGGTCTACACCACCCCAAAGAAGATCAAGCACAAGCACAAGAAGGTCA |
| ODN 5 | SEO1-1262P TTTTTTTTTTGTATGGTTTATTTGATGCTTACTGGTATTATTGCAGATAAATTACACTCT | |
| ODN 6 | YDR411C-894P | TTTTTTTTTTGTCTCAAACCAGTGGCACAGATTCAGGCAGAGCTTCTGGAAGTCAATTAA |
| ODN 7 | YEF3-2800P | TTTTTTTTTTGCTTTGTCTAAGGCTTTGAAGGAATTTGAAGGTGGTGTTATTATCATTAC |
| ODN 8 | YNL208W-542P | TTTTTTTTTTCCTCAGGAATTCGGGGGCCAAGGTCGTCAAGGATTCAATGGCGGTTCACG |

1. A biosensor, comprising:
an electrically or electronically insulating support (2), comprising at least one useful face (20),
a multiplicity of electrically or electronically conducting electrodes (31, 32) that are placed on the useful face (2a) of the support in any predetermined operating arrangement, and are exposed in the sense that said electrodes may be brought together into contact with one and the same external medium, for example liquid,
a plurality of ligands each multiply attached to respectively different electrodes (31, 32),
a multiplicity of electrical terminals (4), corresponding to said electrodes (31) respectively, which are placed on a useful face (2a or 2b) of the support (2) and are exposed in the sense that said terminals may be electrically or electronically connected to the outside independently of one another,
a multiplicity of electrically or electronically conducting tracks (5), each running along one (2a) of the faces of the support (2) and/or the other (2b), connecting the multiplicity of electrodes (31 and 32) to the multiplicity of terminals (4) respectively, and
a layer (6) of an electrically or electronically insulating material coating one (2a) face of the support (2) and/or the other (2b), on the one hand at least partly covering said tracks (5)- and on the other hand exposing both the electrodes (31, 32) and the terminals (5), characterized in that, in combination, on the one hand the multiplicity of electrodes (4) is placed in an extreme zone (1a) on the opposite side from another extreme zone in which the electrical terminals (5) are grouped together, and on the other hand the support (2) includes at least one flexible zone (1c) located between the two extreme zones.

2. The biosensor as claimed in claim 1, characterized in that the entire support is flexible.

3. The biosensor as claimed in claim 1, characterized in that the flexible zone can bend about at least one axis having a direction perpendicular to the direction of alignment of the operating arrangement of the electrodes (31, 32) and of the group of electrical terminals (5).

4. The biosensor as claimed in claim 1, characterized in that the support (2) is a flexible sheet made of insulating material.

5. The biosensor as claimed in claim 1, characterized in that each electrode has at least two adjacent ends (31, 32) connected together.

6. The biosensor as claimed in claim 1, characterized in that at least one other electrically or electronically conducting track (7) runs along one (2a) of the faces of the support and/or along the other (2b), between another electrical terminal (8) placed on a useful face (2b) of the support, which terminal is exposed in order to be connected to a reference potential, and an end (8a) covered with a layer (9) of the electrically or electronically insulating material.

7. The biosensor as claimed in claim 6, characterized in that said other conducting track (7) is assigned to the shielding of the arrangement of the electrodes (31, 32).

8. The biosensor as claimed in claim 6, characterized in that two other electrically or electronically conducting tracks (7, 10) run between two other electrical terminals (8, 11) in order to be connected to a reference potential, these being placed on one face (2a) of the support (2) and the other (2b) respectively, and two respective ends (8a, 10a) that are each covered with the electrically or electronically insulating material.

9. The biosensor as claimed in claim 1, characterized in that at least one electrical terminal (4) is placed on the other face (2b) of the support, which is also a useful face, and the track (5) that corresponds to it passes through the thickness of the support (2).

10. The use of a sensor (1) comprising:
an electrically or electronically insulating support (2), comprising at least one useful face (20),
a multiplicity of electrically or electronically conducting electrodes (31, 32) that are placed on the useful face (2a) of the support in any predetermined operating arrangement, and are exposed in the sense that said electrodes may be brought together into contact with one and the same external medium, for example liquid,
a multiplicity of electrical terminals (4), corresponding to said electrodes (31) respectively, which are placed on a useful face (2a or 2b) of the support (2) and are exposed in the sense that said terminals may be electrically or electronically connected to the outside independently of one another,
a multiplicity of electrically or electronically conducting tracks (5), each running along one (2a) of the faces of the support (2) and/or the other (2b), connecting the multiplicity of electrodes (31 and 32) to the multiplicity of terminals (4) respectively, and
a layer (6) of an electrically or electronically insulating material, coating one (2a) face of the support (2) and/or the other (2b), on the one hand at least partly covering said tracks (5) and on the other hand exposing both the electrodes (31, 32) and the terminals (5),
in order to obtain a biosensor.
